(19) United States
(12) Patent Application Publication
(10) Pub. No.: US 2025/0017574 A1
(43) Pub. Date: Jan. 16, 2025

(54) HOLDER FOR IMAGING CELLS FROM A NEEDLE ASPIRATION SAMPLE

(71) Applicant: AQUYRE BIOSCIENCES, INC., Boston, MA (US)

(72) Inventors: Gregory Brian BOWLES, Fenton, MI (US); Nicholas MANCINI, Boston, MA (US)

(21) Appl. No.: 18/711,366

(22) PCT Filed: Nov. 16, 2022
(86) PCT No.: PCT/US2022/079940

§ 371 Date: May 17, 2024

Related U.S. Application Data
(60) Provisional application No. 63/280,464, filed on Nov. 17, 2021.

(51) Int. Cl. A61B 10/00; G01N 1/40

(52) U.S. Cl. CPC A61B 10/0096; G01N 2001/4088

(57) ABSTRACT

A sample filtering and mounting kit with a reservoir sized and configured to fit in a sample holder of an imaging system, the reservoir having an open end; a filter supported over the open end of the reservoir, the filter having a non-reflective surface and pores sized and configured to allow liquid to pass through to the reservoir and to prevent cells from passing through; a port supported by a port body over the filter, the port adapted to receive a liquid sample, the port body being removable; and a transparent cover adapted to be supported over the filter after removal of the port body. The invention also includes method of using the sample filtering and mounting kit.

### CLAIM OF PRIORITY

This application claims the benefit of U.S. Application No. 63/280,464, filed Nov. 17, 2021, the disclosure of which is incorporated herein in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

In order to diagnose a tissue pathology, such as cancer, a tissue sample may be taken from the patient and analyzed for pathological abnormalities. If the region of interest is accessible, a biopsy sample may be obtained by excising the tissue, and the excised tissue may be sliced for viewing under a microscope in a pathology lab remote from the patient.

As an alternative to the excision of a portion of the tissue, a less invasive biopsy sample may be obtained by aspirating tissue from the region of interest through a small needle into a syringe. Instead of obtaining a solid, organized portion of cellular tissue, however, the fine needle aspiration (FNA) process often extracts a combination of fluid and clumps of cells. To prepare the cells for microscopic viewing or other imaging, they must be separated from the liquid and arranged in a plane on a slide or other platform. For example, U.S. Pat. No. 5,240,606 describes a process of filtering cells from a liquid suspension, then transferring the cells from the filter to a glass slide. U.S. Pat. Nos. 9,541,477 and 10,962,454 describe cell sample preparation processes that centrifuge a liquid suspension to obtain a three-dimensional collection of cells, solidify the three-dimensional cell pellet (e.g., with paraffin or other matrix material), then section the pellet for imaging or other histological examination, e.g., in a pathology lab remote from the patient.

Techniques to obtain a more immediate evaluation of a biopsy sample have been developed. For example, the Aquyre Biosciences CelTivity system employs full-field optical coherence tomography and dynamic cell imaging to perform patient-side evaluation of a tissue sample prior to sending the sample to a pathology lab. Details of these imaging approaches may be found, e.g., in U.S. Pat. Nos. 6,940,602 and 10,627,613. Such imaging systems have been used to image solid tissue samples, not for FNA samples made up of a suspension of cells in liquid.

Rapid on-site evaluation (ROSE) techniques have been developed to determine the adequacy of a FNA biopsy sample by looking for sufficient numbers of target cells in the sample while the patient is still available for additional FNA samples, if needed. The portion of the biopsy sample used in the ROSE evaluation is discarded, however, before the sample is forwarded to the pathology lab.

### SUMMARY OF THE DISCLOSURE

One aspect of the present invention provides a sample holder that can prepare a FNA sample for use in a full-field optical coherence tomography system or a dynamic cell imaging system (such as the Aquyre Biosciences CelTivity system). This aspect of the invention provides a sample filtering and mounting kit including a reservoir sized and configured to fit in a sample holder of an imaging system, the reservoir having an open end; a filter having a non-reflective surface (e.g., a black filter) supported over the open end of the reservoir, the filter having pores sized and configured to allow liquid to pass through to the reservoir and to prevent cells from passing through; a port supported by a port body over the filter, the port adapted to receive a liquid sample, the port body being removable; and a transparent cover adapted to be supported over the filter after removal of the port body.

In some embodiments of the kit, the port includes a luer lock. In some embodiments, the port body is transparent. In some embodiments, the filter is disposed against a bottom surface of the cover.

Some embodiments also include a porous platform disposed at the open end of the reservoir and supporting the filter. Some such embodiments also include a spring biasing the porous platform and filter away from the reservoir to press the filter against a bottom surface of the cover.

Another aspect of the invention provides a method of collecting a cell sample for viewing in an imaging device. In some embodiments, the method includes the steps of: injecting liquid containing the cell sample into a port of a sample filtering device and onto a filter having a non-reflective surface (e.g., a black filter) in the sample filtering device; collecting cells of the cell sample on the filter while passing the liquid through the filter into a reservoir of the sample filtering device; and mounting the sample filtering device in a sample holder of an imaging system so that the cells on the filter can be imaged by the imaging system.

In some embodiments, the sample filtering device includes a port body supporting the port, and the method includes the step of removing the port body after the injecting step and before the mounting step and placing a transparent cover over the filter. In some such embodiments, the method also includes the step of moving the filter against the transparent cover.

In some embodiments, the method includes the step of viewing cells on the filter and repeating the injecting and collecting steps to collect additional cells on the filter before the mounting step. In some such embodiments, the viewing step includes the step of viewing the filter through a transparent port body supporting the port. In some embodiments, the viewing step is performed without further processing of the cells collected on the filter. The method may also include the step of sending the filter and cells to a laboratory for further analysis of the cells.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an exploded view of a sample holder kit according to an embodiment of this invention.

FIG. 2 is a side view of the sample holder of FIG. 1 fitted with an inlet port.

FIG. 3 is an exploded view of the platform and filter paper of the sample holder of FIGS. 1 and 2.

FIG. 4 is a perspective view of the underside of the platform of the sample holder of FIGS. 1 and 2.

FIG. 5 is a schematic view showing the sample holder of this invention in an imaging system.

FIG. 6 is a partial schematic view showing the sample holder of this invention in the imaging system of FIG. 5.

### DETAILED DESCRIPTION

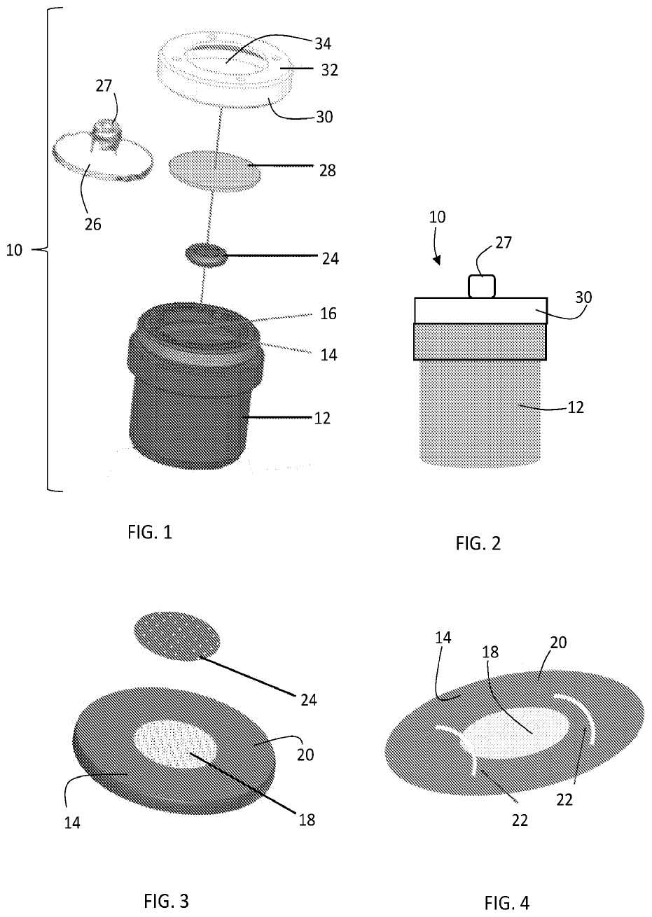

FIGS. 1-4 show a sample holder 10 according to aspects of this invention. The sample holder has a reservoir body 12 supporting a platform 14 within a center ring 16. Platform 14 has a porous portion 18 (shown in FIGS. 3 and 4) surrounded by a ring of support material 20. The porous portion 18 supports a piece of removable filter paper 24. Porous portion 18 has openings large enough to permit water and other liquid to pass through. Platform 14 may be biased upward using, e.g., one or more springs 22.

Filter paper 24 may be opaque and non-reflective (e.g., non-reflective black) to facilitate imaging, as discussed below. Porous portion 18 may be recessed on its top side with respect to ring 20 to hold the filter paper 24 in place. Filter paper 24 may have pores ranging from 1 μm to 20 μm. Alternatively, a solid filter (e.g., plastic or metal) may be used instead of filter paper. The filter medium and pore size may be selected depending on the cells or biologic components of interest and the nature of the liquid, cells, or other biologic components the user wishes to be separated from the cells or biologic components of interest.

The sample holder may have either a transparent sample introduction cover 26 or a transparent cover glass 28 disposed within center ring 16 over the top sides of filter paper 24 and platform 14, as explained below. Sample introduction cover 26 functions as a port holder with an inlet port 27 shaped as a luer lock to interface with the syringe of an FNA needle. A retaining ring 30 having a circular flange 32 and a central opening 34 may be attached to reservoir body 12 via, e.g., threads on the interior of retaining ring 30 and on the exterior of center ring 16. Opening 34 permits a user or imaging system to view the filter paper through either the cover glass 28 or sample introduction cover 26. Springs 22 press platform 14 and filter paper 24 toward the underside of cover 26 or cover glass 28, and retaining ring 30 maintains the cover 26 or cover glass 28 in position so that the filter paper 24 can be pressed firmly but gently.

To prepare an FNA sample for imaging, the sample holder is assembled with the sample introduction cover 26 in place, as in shown in the configuration of FIG. 2. The outlet of a syringe containing a sample obtained from an FNA biopsy may be attached to inlet port 27, and the contents of the syringe may be expressed through inlet port 27 onto filter paper 24. The liquid component of the sample will pass through filter paper 24 and porous portion 18 into reservoir body 12 while the cells within the sample will be retained on the top side of the filter paper 24. The transparent nature of the sample introduction cover 26 enables a user to determine whether a sufficient quantity of cells have been collected by filter paper 24. If not, additional samples can be expressed through input port 27 onto filter paper 24.

After the sample has been injected into the sample holder, retaining ring 30 is removed, sample introduction cover 26 is replaced by cover glass 28, and the retaining ring 30 is re-applied to holder cover glass 28 firmly against the top side of filter paper 24. Springs 22 ensure that the filter paper 24 and the cells it retained are pressed against the underside of cover glass 28 to, e.g., distribute the cells in a thin layer and to minimize optical interference.

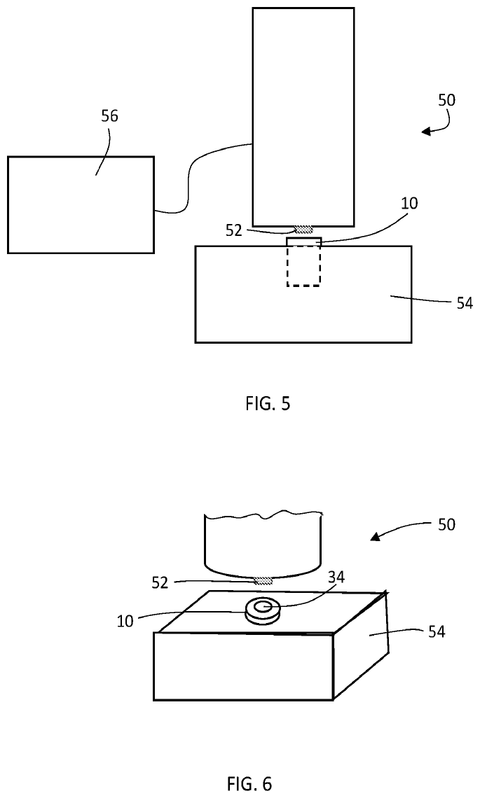

The sample is then ready for imaging by an imaging system. An imaging system 50 is shown in FIGS. 5 and 6. Imaging system 50 may be, e.g., the Aquyre Biosystems CelTivity system or any other suitable imaging system, such as those described in U.S. Pat. Nos. 6,940,602 and 10,627,613. As shown, imaging system 50 has, among other components, imaging hardware including an imaging lens 52 transmitting imaging light, a sample platform 54, imaging electronics (not shown), and a display 56.

Sample holder 10 may be loaded into the sample platform 54 of imaging system 50, and the cells on filter paper 24 may be imaged by the imaging system 50 without any further processing of the sample. The opaque, non-reflective nature of filter paper 24 (e.g., non-reflective black) facilitates the imaging. After imaging the cells, the sample holder with the cells on the filter paper 24 and the liquid component of the sample in reservoir 12 may be transferred to a pathology lab or elsewhere for further analysis.

The sample holder of this invention, when used in connection with an imaging system such as the Aquyre Biosystems CelTivity system, enables all of the cells collected in the FNA biopsy to be imaged. By contrast, the ROSE procedure images only a fraction of the sample, usually only one drop, and the remaining portion of the sample is often not imaged for days.

In addition, the sample holder of this invention preserves the entire sample—the cells on the filter and the liquid they had been suspended in—for subsequent analysis by a pathologist. The drop of sample used in the ROSE procedure, on the other hand, is discarded and is never seen by the pathologist. Furthermore, the sample holder of this invention enables the sample to be imaged without any further processing (e.g., without stains, dyes, fixative agents or physically sectioning the sample).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A sample filtering and mounting kit comprising:
a reservoir sized and configured to fit in a sample holder of an imaging system, the reservoir having an open end;
a filter supported over the open end of the reservoir, the filter having a non-reflective surface and pores sized and configured to allow liquid to pass through to the reservoir and to prevent cells from passing through;
a port supported by a port body over the filter, the port adapted to receive a liquid sample, the port body being removable; and
a transparent cover adapted to be supported over the filter after removal of the port body.

2. The kit of claim 1, wherein the port comprises a luer lock.

3. The kit of claim 1, wherein the port body is transparent.

4. The kit of claim 1, wherein the filter is disposed against a bottom surface of the cover.

5. The kit of claim 1, further comprising a porous platform disposed at the open end of the reservoir and supporting the filter.

6. The kit of claim 5, further comprising a spring biasing the porous platform and filter away from the reservoir to press the filter against a bottom surface of the cover.

7. The kit of claim 1, wherein the filter is black.

8. A method of collecting a cell sample for viewing in an imaging device, the method comprising:
injecting liquid containing the cell sample into a port of a sample filtering device and onto a non-reflective surface of a filter in the sample filtering device;
collecting cells of the cell sample on the filter while passing the liquid through the filter into a reservoir of the sample filtering device; and
mounting the sample filtering device in a sample holder of an imaging system so that the cells on the filter can be imaged by the imaging system.

9. The method of claim 8, wherein the sample filtering device further comprises a port body supporting the port, the method further comprising removing the port body after the injecting step and before the mounting step and placing a transparent cover over the filter.

10. The method of claim 9, further comprising moving the filter against the transparent cover.

11. The method of claim 8, further comprising viewing cells on the filter and repeating the injecting and collecting steps to collect additional cells on the filter before the mounting step.

12. The method of claim 11, wherein the viewing step comprises viewing the filter through a transparent port body supporting the port.

13. The method of claim 11, wherein the viewing step is performed without further processing of the cells collected on the filter.

14. The method of claim 11, further comprising sending the filter and cells to a laboratory for further analysis of the cells.
